(19) United States
(12) Patent Application Publication
(10) Pub. No.: US 2008/0082119 A1
(43) Pub. Date: Apr. 3, 2008

(54) Method of determining an appropriate catheter length

(76) Inventor: Jeffrey M. Vitullo, Sinking Spring, PA (US)

Correspondence Address: AMSTER, ROTHSTEIN & EBENSTEIN LLP, 90 PARK AVENUE, NEW YORK, NY, 10016, US

(21) Appl. No.: 11/542,051

(22) Filed: Oct. 3, 2006

(51) Int. Cl. A61M 29/00

(52) U.S. Cl. 606/192

(57) ABSTRACT

A method of inserting a catheter into a body of a patient includes identifying a catheter insertion site on the patient's body; measuring a length, from the insertion site to another site external to the body of the patient, and along an external path that follows a presumed internal path of the catheter from the insertion site to a target site within the patient's body, using an automatic measuring device; trimming the catheter to an appropriate length based on the length measured by the automatic measuring device; and inserting the trimmed catheter into the body of the patient through the insertion site to the target site.

### FIELD OF THE INVENTION

The present invention relates to a method of measuring the appropriate length of a medical device to be inserted into the body of a patient, and more particularly to a method for measuring a portion of a patient's body to determine the appropriate length of a catheter to perform a medical procedure.

### BACKGROUND OF THE INVENTION

Proper tip placement for a catheter within the body of a patient is usually achieved by measuring some external anatomical feature and choosing and/or trimming the catheter to be placed to the proper length based on this anatomical measurement. Typically, a paper measuring tape is included in the procedure tray along with all of the other disposable equipment utilized in placing a catheter. This measuring tape is used to measure the desired anatomical feature to determine the appropriate length of the catheter to be inserted.

A clinician can not always accurately measure an anatomical feature using a paper measuring tape, and manipulating the tape along the patient's body to obtain a measurement is often an awkward and time-consuming chore. Accordingly, there is a need for a relatively simple method for accurately and quickly measuring an anatomical feature to determine an appropriate catheter insertion length.

### SUMMARY OF THE INVENTION

A method of inserting a catheter into a body of a patient according to an exemplary embodiment of the present invention includes identifying a catheter insertion site on the patient's body; measuring a length, from the insertion site to another site external to the body of the patient, and along an external path that follows a presumed internal path of the catheter from the insertion site to a target site within the patient's body, using an automatic measuring device; trimming the catheter to an appropriate length based on the length measured by the automatic measuring device; and inserting the trimmed catheter into the body of the patient through the insertion site to the target site.

A method of inserting a catheter into a body of a patient according to another exemplary embodiment of the present invention includes identifying a catheter insertion site on the patient's body; measuring a length, from the insertion site to another site external to the body of the patient, and along an external path that follows a presumed internal path of the catheter from the insertion site to a target site within the patient's body, using an automatic measuring device; and inserting the catheter into the body of the patient through the insertion site by an amount determined based on the length measured by the automatic measuring device.

A method of determining appropriate length of a catheter to be inserted into a body of a patient according to an exemplary embodiment of the present invention includes identifying a catheter insertion site on the patient's body; and measuring a length, from the insertion site to another site external to the body of the patient, and along an external path that follows a presumed internal path of the catheter from the insertion site to a target site within the patient's body, using an automatic measuring device.

An automatic measuring device for measuring length of an anatomical feature according to an exemplary embodiment of the present invention includes a first portion including a measuring tool that generates signals when placed in contact with and traversed along the anatomical feature, and a second portion including a processor that processes the generated signals to produce data representing the length of the anatomical feature.

These and other features of this invention are described in, or are apparent from, the following detailed description of various exemplary embodiments of this invention.

### BRIEF DESCRIPTION OF THE DRAWINGS

Various exemplary embodiments of this invention will be described in detail, with reference to the following figures, wherein:

FIG. 1 shows a wheeled length measuring device useable with an exemplary embodiment of the present invention;

FIG. 2 is a partial block diagram of the wheeled length measuring device of FIG. 1;

FIG. 3 shows a measuring device according to an exemplary embodiment of the present invention; and

FIG. 4 is a flow chart illustrating a method of inserting a catheter into a body of a patient according to an exemplary embodiment of the present invention.

### DETAILED DESCRIPTION OF PREFERRED EMBODIMENTS

According to exemplary embodiments of the present invention, a measuring device is used to determine the appropriate length of a medical device required to perform a medical procedure. For example, the measuring device may be used to estimate the length of a catheter required to reach from an insertion site to a target site within the patient's body. According to an exemplary method of the invention, a measuring device is used to estimate the distance from a catheter insertion site to a target site by running the measuring device along and external to the body of the patient to follow a presumed path from the insertion site to the target site. The catheter may then be appropriately trimmed according to the estimated distance obtained by the measuring device. Although a measuring device that uses a rotating measuring wheel is described herein, it should be appreciated that any suitable automatic measuring device may be used in the present inventive method. Further, the measuring wheel may be either an analog or a digital device.

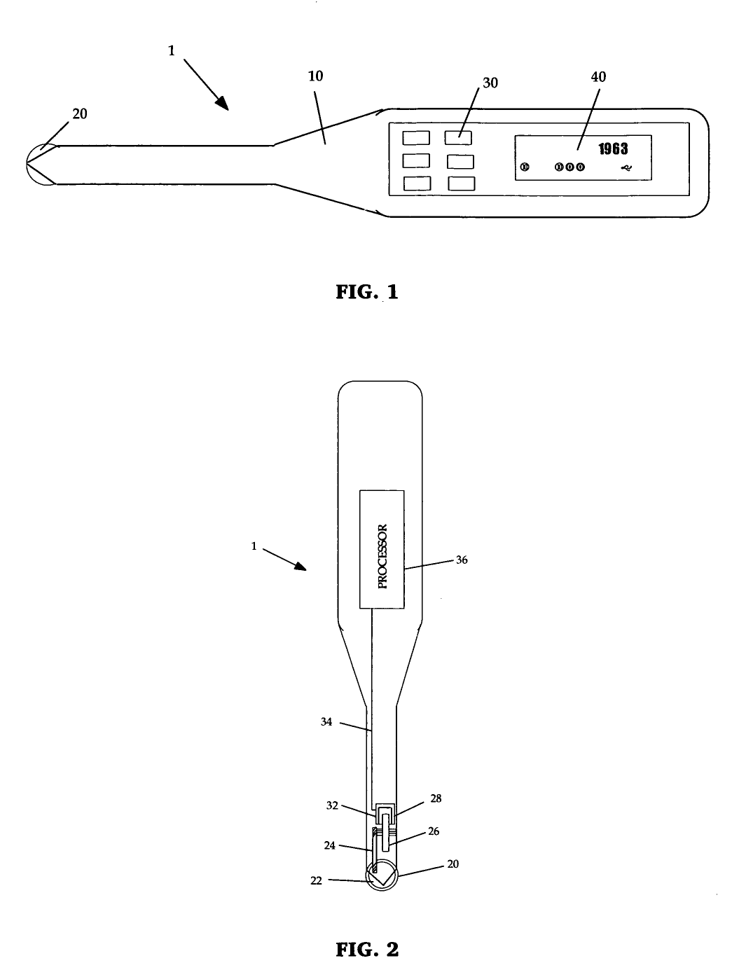

FIG. 1 shows a wheeled measuring device, generally designated by reference number 1, useable with a method of determining the appropriate length of a catheter according to an exemplary embodiment of the present invention. Such wheeled measuring devices are well known to engineers and cartographers, and the measuring device described herein is merely exemplary of the measuring device that may be used in the present invention. An example of a suitable digital measuring device useable with the present invention is the PlanWheel™, manufactured by Scalex Corporation, of Carlsbad, Calif. An analog measuring device may include a click counter that counts the number of rotations of a wheel along a path to determine the length of the path. Such click counting technology is known from surveying instruments, for example.

The measuring device 1 includes a housing 10 that is preferably pen-shaped to fit comfortably within one hand of a user. The housing 10 is preferably made of a rigid and lightweight material, such as, for example, plastic. A measuring wheel 20 is mounted at a distal end of the housing 10. As explained in further detail below, the measuring wheel 20 may be rolled along a straight or curved path so that the measuring device 1 can determine the length of the path. The measuring device 1 further includes a number of function keys 30 that allow a user to perform calculations using the length data collected by the measuring device 1, such as, for example, calculations of surface area and scaling operations. In this regard, the measuring device 1 includes a display screen 40 that displays length data and calculation results.

FIG. 2 is a partial block diagram of the measuring device 1. The measuring wheel 20 is mounted at the distal end of the measuring device 1 for rotation about an axis perpendicular to the longitudinal axis of the housing 10. The gear wheel 22 is disposed on one face of the measuring wheel 20. When the measuring wheel 20 rotates, the gear wheel 22 drives a shaft 24, which in turn drives a disk 26. A light-generating element 28 is disposed at one side of the disk 26, and a signal-receiving element 32 is disposed at an opposite side of the disk 26. The disk 26 is provided with a number of narrow slots or holes (not shown) at regular intervals. The slots in the disk 26 create a pulse train by alternately allowing the passage of a light beam emitted from the light-generating element 28. The light beam passes to the signal-receiving element 32, which converts the pulse train into an input signal. The input signal is passed through a signal line 34 to a processor 36, which uses the input signal to calculate the distance traveled by the measuring wheel 20. The processor 36 may also perform other calculation functions as needed based on user input through the function keys 30.

FIG. 3 shows a measuring device, generally designated by reference number 100, according to another exemplary embodiment of the present invention. The structure of the measuring device 100 is essentially the same as that of the previous embodiment except that the distal end of the measuring device 100, including the measuring wheel 20, may be replaceable for hygienic purposes. In particular, the measuring device 100 may include separable first and second portions 110, 120 that are connected to one another by, for example, a snap lock connection. The measuring wheel 20 is disposed at the distal end of the second portion 120, and an electrical contact 122 is disposed at a proximal end of the second portion 120. The electrical contact 122 engages with an electrical contact 112 disposed at the distal end of the first portion 110, so that measurement signals from the second portion 120 can be sent to the processor 36 located within the first portion 110. As an example, the contacts 122 and 112 may be a plug and socket pair.

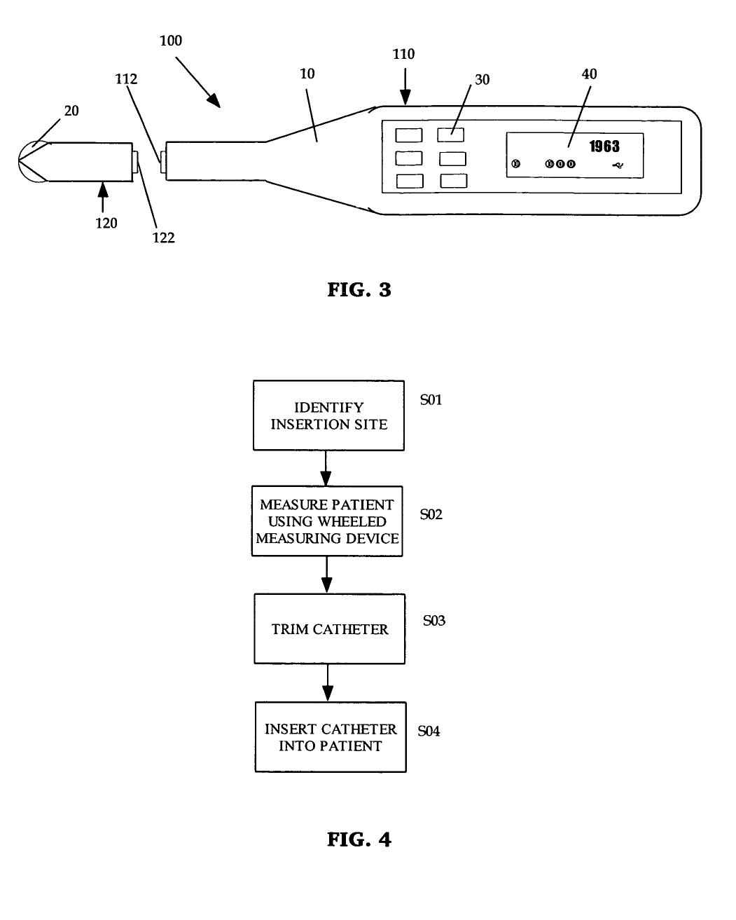

FIG. 4 is a flow chart illustrating a method of inserting a catheter into a body of a patient according to an exemplary embodiment of the present invention. In step S01, the proper location of the insertion site is identified. For example, a peripherally inserted central (PIC) catheter is typically inserted into basilica, medial cubital or cephalic veins of an adult. In step S02, the patient is measured using the measuring device 1 to determine the appropriate length of the catheter. In particular, the measuring wheel 20 may be rolled along a portion of the patient's body to measure the distance from the insertion site to the target site along the presumed anatomical course of the vessel to be catheterized. In this step, the measuring device 1 may be in communication with a printer so that the length of the catheter may be printed out for documentation purposes. In step S03, the catheter is trimmed to the length as measured by the measuring device 1 using an appropriate trimming device. In step S04, the trimmed catheter is inserted into the insertion site and passed to the target site.

In embodiments of the present invention, the measured length of the catheter may be modified by the user. For example, if a catheter securement device is used, 1 to 1½ inches may be added to the length measurement. The added length may be done manually, or the alternatively the measuring device 1 may be programmed to add the additional length once the base measurement is determined.

According to another exemplary embodiment of the present invention, the length measurement obtained by the measuring device may be used to determine the appropriate amount of the catheter to be inserted into the patient to reach the target site without trimming of the catheter.

While this invention has been described in conjunction with the exemplary embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

What is claimed is:

1. A method of inserting a catheter into a body of a patient, comprising:
identifying a catheter insertion site on the patient's body;
measuring a length, from the insertion site to another site external to the body of the patient, and along an external path that follows a presumed internal path of the catheter from the insertion site to a target site within the patient's body, using an automatic measuring device;
trimming the catheter to an appropriate length based on the length measured by the automatic measuring device; and
inserting the trimmed catheter into the body of the patient through the insertion site to the target site.

2. The method of claim 1, wherein the automatic measuring device comprises a measuring wheel, and the step of determining a length comprises moving the measuring wheel along the external path.

3. The method of claim 1, wherein the automatic measuring device is a digital device.

4. The method of claim 1, wherein the automatic measuring device is an analog device.

5. The method of claim 1, further comprising placing the automatic measuring device in communication with a printer to print out length data including the measured length.

6. A method of inserting a catheter into a body of a patient, comprising:
identifying a catheter insertion site on the patient's body;
measuring a length, from the insertion site to another site external to the body of the patient, and along an external path that follows a presumed internal path of the catheter from the insertion site to a target site within the patient's body, using an automatic measuring device; and
inserting the catheter into the body of the patient through the insertion site by an amount determined based on the length measured by the automatic measuring device.

7. The method of claim 6, wherein the automatic measuring device comprises a measuring wheel, and the step of determining a length comprises moving the measuring wheel along the external path.

8. The method of claim 6, wherein the automatic measuring device is a digital device.

9. The method of claim 6, wherein the automatic measuring device is an analog device.

10. A method of determining appropriate length of a catheter to be inserted into a body of a patient, comprising:
identifying a catheter insertion site on the patient's body; and
measuring a length, from the insertion site to another site external to the body of the patient, and along an external path that follows a presumed internal path of the catheter from the insertion site to a target site within the patient's body, using an automatic measuring device.

11. The method of claim 10, wherein the automatic measuring device comprises a measuring wheel, and the step of determining a length comprises moving the measuring wheel along the external path.

12. The method of claim 10, wherein the automatic measuring device is an analog device.

13. The method of claim 10, wherein the automatic measuring device is a digital device.

14. An automatic measuring device for measuring length of an anatomical feature, comprising:
a first portion including a measuring tool that generates signals when placed in contact with and traversed along the anatomical feature; and
a second portion including a processor that processes the generated signals to produce data representing the length of the anatomical feature.

15. The measuring device of claim 1, wherein the measuring tool is a measuring wheel.

16. The measuring device of claim 1, wherein the first portion is separable from the second portion.
